(19) United States
(12) Patent Application Publication
(10) Pub. No.: US 2007/0003490 A1
(43) Pub. Date: Jan. 4, 2007

(54) Medicated gumstick for treatment in anti-inflammatory conditions and prophylaxis against NSAID gastropathy

(75) Inventor: Pardeep Nijhawan, Richmond Hill (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: MEDICAL FUTURES INC.

(21) Appl. No.: 11/168,526

(22) Filed: Jun. 29, 2005

(51) Int. Cl. A61K 9/68

(52) U.S. Cl. 424/48

(57) ABSTRACT

A stick of gum is provided containing therapeutic benefits of non-steroid anti-inflammatory drugs for inflammation in conditions such as arthritis, and also alleviates subsequent side effects of NSAID administration, as well as antacid effects from compounds such as an H2 antagonist (ranitidine, cimetidine, famotidine) and/or a proton pump inhibitor (such as lansoprazole, pantoprazole, omeprazole, esomeprazole or rabeprazole) and/or an acid pump antagonist selected from the group of soraprazan, AZD0865, YH1885 and CS-526.

### FIELD OF THE INVENTION

The present invention provides a composition capable of eliminating or reducing the undesirable side effects resulting from NSAID administration. This composition is formulated as a stick chewing gum for convenience and ease of use.

### BACKGROUND OF THE INVENTION

The efficacy of non-steroidal antiinflammatories (NSAIDs) for treatment of pain, inflammation and fever, has made these drugs the most commonly used in the world. Unfortunately, this class has been limited by their gastrointestinal (GI) toxicity, a side effect that has caused numerous problems. These complications can include dyspepsia, GI upset, gastric ulceration, gastric erosion, duodenal ulceration and esophagitis. NSAID gastropathy affects millions of people annually and results in increased hospitalizations and deaths due to complications from gastroduodenal bleeds. Furthermore, the incidence of dyspepsia has been reported to be at 15% in patients taking either NSAIDs or COX-2 inhibitors. These patients require additional medications, such as gastroprotective agents to help reduce the incidence of dyspepsia.

In addition, NSAID induced gastropathy can affect up to 80% of all patients who use NSAIDs chronically. Even in those patients using NSAIDs for the short term, gastropathy can begin within 72 hours of ingestion of the first dose of the medication, leading to gastric mucosa damage. In some cases, patients will arrive at the emergency room with massive upper gastrointestinal tract bleeds that may be life-threatening. In fact, gastrointestinal complications from NSAIDs are the most common single side effect of any class of drugs in America today.

In patients who do develop gastrointestinal bleeding from use of NSAIDs, urgent endoscopy and therapy with a group of drugs known as proton pump inhibitors (PPI) is required. Proton pump inhibitors include lansoprazole, pantoprazole, omeprazole, esomeprazole and rabeprazole. These drugs have replaced the older histamine receptor blockers as the new gastrointestinal protective agent of choice, although H2 antagonists still play a major role in prevention of NSAID gastropathy. PPI are useful for treating active ulcers, decreasing the incidence of recurrence of ulcers, decreasing gastric and esophageal inflammation and decreasing the incidence of gastrointestinal bleeding. PPIs and H2 antagonists are collectively referred to as gastroprotective agents (GPA).

NSAID induced gastropathy can affect all patients including otherwise young and health adults. However, of the great number of patients requiring NSAID therapy, certain groups are at increased risk of developing the GI complications. These groups include those of advanced age and co morbid conditions such as significant heart disease or rheumatoid arthritis. Unfortunately, it is in these groups that relief of inflammatory conditions would be most desired and appreciated.

Adding to these increased complications, there is suggestion from several reports that as we age, we are more likely to use NSAIDs and also other medications. These medications can lead to an increase in the incidence of pill induced esophagitis. Once again, this can be prevented by using a GPA when ingesting pills.

These complications formed the basis for intensive research on the inflammatory state, and it was in the early 1990s that a solution to this problem presented itself with the identification of two structurally related cyclo-oxygenase isoforms. Labelled COX-1 and COX-2, it was the COX-2 isoform that was identified as the principal isoform involved in inflammation. This concept led to the development and clinical introduction of COX-2-specific NSAIDs, drugs noted for their reduced GI toxicity.

Unfortunately, after a decade of use, retrospective analysis confirmed the incidence of increased cardiac toxicities associated with long term use of COX-2 NSAIDs. This realization has recently prompted a return to the use of traditional non-selective NSAIDs for treatment of inflammation. With this return a rise in the occurrence of NSAID GI complications is anticipated.

In light of the clinical failure of the COX-2 selective NSAIDs for long-term treatment of inflammatory conditions, there is a need once again for the relief provided by traditional NSAID therapy. At the same time there is a dire need for treatment of the aforesaid harmful side effects arising from NSAID therapy. This urgent and long felt need has been met with the present invention.

### PRIOR ART

Within the prior art there are several compositions formulated including tablets and also chewing gum formulations. All of these fail to address the unique benefits achieved by the present invention.

The prior art includes U.S. Pat. No. 5,037,815 by Lukacsko et al, wherein NSAIDs are combined with a different group of gastroprotective agents, including H2 antagonists. This patent outlines how an NSAID and H2 antagonists can be combined in a tablet formulation. However, it does not include a gum formulation for increased salivation and increased buccal mucosa absorption for improved treatment of migraine headaches.

U.S. Pat. No. 6,537,525 describes a chewing gum composition useful for treatment for gastroesophageal reflux disease. The composition is taught to include at least one ingredient from the group consisting of an acid neutralizing agent, and anti-gas agent, and an acid production inhibitor. Acid production inhibitors include H2 receptor antagonists and PPIs, as taught within this patent. Examples of H2 receptor antagonists are listed as cimetidine, ranitidine and famotidine. Examples of PPIs are listed as lansoprazole and omeprazole. However, the advantage of these agents combined with an NSAID for delivery with the chewing gum composition is not contemplated, nor are the benefits taught of a distinct separation of active ingredients within the chewing gum. The stick of gum of the present invention is unique in that it aims primarily to relieve inflammatory conditions using an NSAID, while simultaneously ameliorating consequences of such a therapeutic regimen through the inclusion of GPAs.

U.S. Pat. No. 6,773,716 relates to a chewing gum product containing a medicament in the coating. The medicament is selected from a group consisting of antacids and anti-inflammatories, including famotidine and omeprazol, and indomethacin and ibuprofen. The present invention addresses the need for a chewing gum stick which allows for ease of use (as opposed to blister packs) in the affected arthritic population while provided enhanced absorption through the buccal mucosa from a larger surface area. Furthermore, the immediate invention does not formulate a medicament in the coating, but rather is unique in the placement and separation of active ingredients in the chewing gum stick, designed to enhance stability.

WO 2004/004478 teaches a compressed chewing gum tablet, and sets out methods for encapsulating the chewing gum centre fully or partly with a barrier layer. The art teaches methods of layering gum tablets outlined in the disclosure, along with an improved method for obtaining a barrier layer. This barrier layer is taught to protect the gum centre during manufacture. Although this patent application contemplates different pharmaceutical ingredients including acetaminophen, ibuprofen, and indomethacin, along with other NSAIDs, ranitidine and other H2 agonists, and lansoprazole and other PPIs, these are formulated in a tablet. The present invention is formulated to provide enhanced absorption, and ease of use in the target population through formulation as a soft stick of gum.

WO 2004/068965 relates to a chewing gum tablet characterized by at least two individual chewing gum modules, that is, physically distinct layers, such that degradation of the products through side reactions may be avoided. This is accomplished through a particular combination of polymers used for each gum base. In one embodiment of the invention, the modules are tablet slice-like layers, each having different ingredients intended to be separated in the tablet. The present invention is not characterized as a chewing gum tablet with distinct layers; rather, it is formulated as a soft stick for ease of use in the target population suffering from inflammatory conditions.

US patent application 2002/0164398 teaches a chewing gum product exhibiting accelerated absorption of medicaments through oral mucosa. There is taught a chewing gum product containing at least one medicament in the coating, with a water-soluble alkaline material incorporated in the centre. Medicaments, as used in this composition, are taught to include antacids, such as omeprazole and famotadine (among others), and anti-inflammatories, such as indomethacin or ibuprofen. This US application fails to address the need for a soft chewing gum stick incorporating the present invention.

The above-mentioned teachings are inadequate for alleviation of both inflammation and, subsequent NSAID gastropathy effectively in the manner taught in the present application and fail to recognize the distinct needs of the target population. As previously mentioned, a large portion of those at risk of NSAID-induced GI complications include those who are elderly and/or arthritic. These conditions can complicate the otherwise simple use of chewing gums formulated and delivered as tablets in blister packs. The difficulty in making use of such a delivery is certainly one that can be appreciated by those suffering from an arthritic state. The unique nature of those individuals who require NSAID therapy, but are unable to cope with the detrimental side effects, demands a unique and inventive therapeutic approach. The present invention has met this need with a soft chewing gum formulated delivered as a soft stick.

However, in spite of the general discussions in the above-mentioned patent literature there is no discussion of the problems facing the elderly in handling blister packages or the like. Blister packages require a certain amount of strength and effort potentially beyond patients suffering from arthritis in the joints. The manual dexterity required to open and consume such packaging can be an inhibiting factor for those in dire need of the required medicaments.

It is therefore a primary object of this invention to provide a stick gum based pharmaceutical which is easy to use.

It is a further object of this invention to provide the stick gum based product which includes two separate components separated by an enteric layer.

It is a further object of this invention to provide the stick gum based product which includes an NSAID component and a PPI component or H2 antagonist component separated by an enteric layer.

Further and other objects of the invention may become apparent to those skilled in the art when considering the following summary of the invention and a more detailed description of the preferred embodiments illustrated herein.

### SUMMARY OF THE INVENTION

The current invention allows for the combination of GPA and an NSAID in a single stick of gum formulated for chronic use by high risk patients. This novel delivery method allows for ease of use, increased compliance and decreased adverse events of NSAID therapy. In particular, it is envisioned that this formulation will provide high risk patients with a treatment option that is more conducive to the conditions experienced in their clinical state.

More particularly, a gum based stick of gum composition is provided that is useful in having the therapeutic benefits of non-steroid anti-inflammatory drugs for inflammation in conditions such as migraine and arthritis and also includes antacid effects from compounds such as an H2 antagonist (ranitidine, cimetidine, famotidine) and/or a proton pump inhibitor (such as lansoprazole, pantoprazole, omeprazole, esomeprazole or rabeprazole). These antacids, generally referred to as GPAs, help decrease the incidence of gastrointestinal complications in patients taking NSAIDs. These complications may include dyspepsia, gastrointestinal upset, gastric ulceration, gastric erosion, duodenal ulceration, and esophagitis.

As many active substances are lipophilic, they will adhere to the gum base and may therefore be released slowly and incompletely. Methods to increase rate and extent of the release include the addition of buffering agents or solubilizing agents and coating/encapsulation of active substances.

In contrast, hydrophilic active substances are rapidly released and it may therefore be necessary to slow down the release rate by means of various methods, e.g. by encapsulating the active substance or by increasing the amount of gum base.

Local Effect

Chewing gum is a useful drug delivery system for local treatment of diseases in the oral cavity and in the throat, as exposure may be deliberately prolonged by sustaining the release of active substances. Generally, chewing gum is chewed for a longer period of time than chewable tablets, thus enabling a longer exposure time. Compared to lozenges, chewing gum allows for much better control of the release rate, even though chewing gum is chewed with different intensity and at different chewing rates. This is mainly because lozenges are often unintentionally chewed.

Systemic Effect

Active substances can be absorbed through the buccal mucosa and/or through the gastrointestinal tract when saliva is swallowed. Once the active substance is present in the blood, systemic effects can be obtained.

Fast Onset of Action

Fast onset of systemic effect is seen for active substances absorbed through the buccal mucosa, as the active substances pass by the jugular veins directly to the systemic circulation. Chewing gum is an ideal drug delivery system for active substances with buccal absorption. For substances with low or no buccal absorption, fast onset of action may still be achieved as the substances are already dissolved or suspended in the saliva by the time they reach the gastrointestinal tract. This is ideal for treatment in conditions such as migraines.

Fewer Side Effects

Active substances absorbed buccally bypass the hepatic first pass metabolism, which may result in a higher bioavailability of the active substance. Thus, the equivalent efficacy may be obtained with a lower dosage, and consequently fewer side effects are expected. Further, a lower dosage may reduce the risks of interactions with other active substances. The controlled release rate also reduces the risk of side effects, as high plasma peak concentrations are avoided.

Less Risk of Overdosing

Chewing is required to release the active substance from chewing gum. If the chewing gum is swallowed accidentally, only limited amounts of the active substance will be released over a relatively long period of time, thus reducing the risk of high plasma peak concentrations and overdosing.

Effect on Dry Mouth (Xerostomia)

Dry mouth is a side effect of many types of medication (e.g. antidepressants), and it is also part of several diseases (e.g. Sjögren's syndrome). It is well known that chewing gum stimulates salivary secretion and a chewing gum formulation therefore partly alleviates this condition. Furthermore, as dry mouth increases the incidence of dental caries, chewing gum may also be beneficial to dental health. It has been shown that long-term activation of the salivary glands by chewing gum several times per day for two months enhanced resting salivary flow, especially in individuals with low salivary flow

Patient Compliance

As no water is required, taking medication in chewing gum is very convenient and therefore suitable for acute treatment. The medication may be taken without regard to time and place, thus promoting compliance. Chewing gum does not draw attention to the medication; it is discrete and does not stigmatize the patient. Today, there is a trend towards higher patient involvement in drug administration and handling. Chewing gum is in line with this trend as it allows easy self-administration and does not prevent patients from living an active life.

### SUMMARY

In summary, the medicated chewing gum allows for the two active drugs (GPA and NSAID) to be delivered in an effective manner that is pleasant tasting, convenient, improves compliances, decreases adverse events, improves drug delivery while decreasing local toxic effects of NSAIDs, and improves salivation which decreases the incidence of pill induced esophagitis.

Medicated chewing gum has become more noticeable with the variety of products available to help cease smoking. As effective as it has been with cessation of smoking, it has not become a normal delivery method for medications. When scientists have looked at products for medicated chewing gum in the past, it has involved poor drug availability and/or poor taste. In the past several attempts at using NSAIDs in chewing gums have failed due to the poor taste.

This current invention addresses the need for a separate delivery mechanism for NSAIDs in the format of a chewing gum. This chewing gum stick product will have two gum sections, one containing an active GPA such as a PPI and or an H2 antagonist and a second section containing an NSAID. The two sections will be separated by an enteric layer that will be filled with flavor, and sweetening agents to help decrease the unpleasant taste of the NSAID.

This layer will also serve to keep the acidic NSAID away from the PPI and or H2 antagonist and thereby decreasing the chances of instability and degradation in the product. This will allow for enhancement of the shelf life of the product and allow for improvement of bioavailability.

The product when consumed, will allow for rapid absorption through the buccal mucosa of the PPI and/or H2 Antagonist and the NSAID. The increased absorption will be aided by the fact that the gum stick will have an increase in surface area. The stick of gum is designed to decrease the area of contact of the two medications to help with bioavailability. This will lead to an improvement in delivery of drug for pain and inflammation in arthritis and migraine suffers. It will also allow the GPA to begin blocking the appropriate receptors so that the amount of acid in the stomach is decreased. Another benefit of this formulation is that there is less contact with the NSAID and the direct lining of the stomach. This will help to decrease the incidence of local ulceration and damage from the NSAID. Furthermore, the chewing effect of the gum allows for improved salivation and peristalsis in the esophagus. Both of these actions help decrease the incidence of NSAID gastropathy. Finally this will also help to keep the incidence of pill induced esophagitis to a minimum.

The GPA agents are able to have rapid absorption through the buccal mucosa. This will allow for the H2 antagonist to have a rapid response to dyspepsia symptoms (within minutes). However, H2 antagonists have a short half life and therefore would stop having clinical effects with a few short hours. By that time, the PPI would become efficacious and allow for the proton pumps in the stomach to be inhibited for longer durations (typical 24 to 36 hours).

According to a primary aspect of the invention there is provided a stick of medicated chewing gum comprising:
(a) a first part containing a gum base and a non-steroidal anti-inflammatory drug (NSAID);
(b) a second part containing a gum base and a protective amount of a proton pump inhibitor (PPI); and or
a protective amount of H2 antagonists; and/or
an acid pump antagonist.

Preferably said NSAID is selected from the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts thereof, and mixtures thereof.

Preferably said PPI selected from a group consisting of lansoprazole, omeprazole, rabeprazole, esomeprazole, or pantoprazole and pharmaceutically acceptable salts thereof preferably present in doses of 0 mg to 40 mg per unit composition being administered.

In one embodiment the active ingredients are embedded within the first and or second parts separated by a calcium layer that contains a flavoring agent. Preferably said flavoring agent is selected from the group consisting of menthol, organic acids, oil of cinnamon, oil of wintergreen, oil of peppermint, oil of spearmint, oil of sassafras, and oil of clove. The gum stick may further comprise an increased surface area to allow for easy absorption via the buccal mucosa, while minimizing contact between the medications. In one embodiment said stick of medicated chewing gum is formulated as a soft chewing stick to facilitate delivery and use by the affected population.

Preferably said stick of gum may further comprise sweetening agents selected from the group consisting of sucrose, glucose, dextrose, levulose, saccharin salts, thaumatin, aspartame, D-tryptophan, dihydrochalcones, acesulfame salts cyclamate salts, sorbitol, xylitol, maltitol and mixtures thereof.

Preferably said H2 agonist is selected from the group consisting of ranitidine, cimetidine, or famotidine and pharmaceutically acceptable salts thereof and is preferably present in doses of 0 mg to 150 mg per unit composition being administered.

In one embodiment said acid pump agonist is selected from the group consisting of soraprazan, AZD0865, YH1885 and CS-526 and pharmaceutically acceptable salts thereof.

### BRIEF DESCRIPTION OF THE FIGURES

FIG. 1 is a top view of the gum stick showing the two preferred components, NSAID and GPA, separated by a calcium layer which contains flavouring.

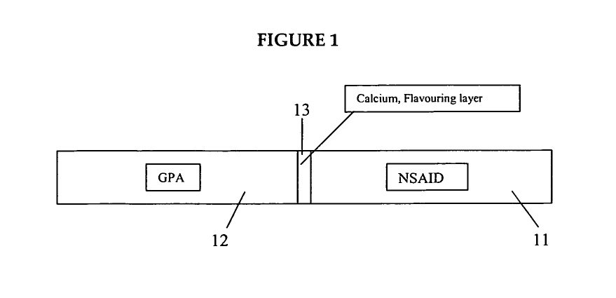

FIG. 2 is the profile view of the gum stick showing the two sections separated by the calcium and flavouring layer.

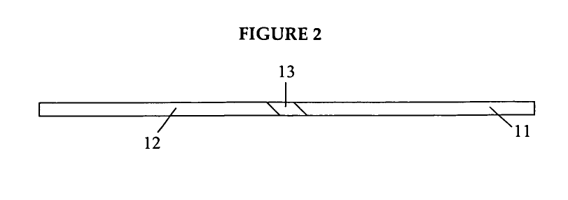

### DETAILED DESCRIPTION OF THE PREFERRED EMBODIMENTS

The stick gum based pharmaceutical composition of the present invention is a medicated chewing gum that reduces the potential for NSAID induced gastropathy and comprises: (a) a non-steroidal anti-inflammatory drug (NSAID) which is selected from the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts thereof, and mixtures thereof; and (b) a protective amount of proton pump inhibitor (PPI) such as lansoprazole, omeprazole, rabeprazole, esomeprazole, or pantoprazole and pharmaceutically acceptable salts thereof; and/or a protective amount of H2 antagonists such as ranitidine, cimetidine, or famotidine and pharmaceutically acceptable salts thereof.

This medicated gum based composition contains a flavoring agent that is selected from the group consisting of menthol, organic acids, oil of cinnamon, oil of wintergreen, oil of peppermint, oil of spearmint, oil of sassafras, and oil of clove.

The medicated gum based composition also contains a sweetening agent that is selected from the group consisting of sucrose, glucose, dextrose, levulose, saccharin salts, thaumatin, aspartame, D-tryptophan, dihydrochalcones, acesulfame salts cyclamate salts, sorbitol, xylitol and maltitol.

The proton pump inhibitor may be present in doses of 0 mg to 40 mg per unit composition being administered.

The active ingredient may be present as follows:
omeprazole in the composition is present in amount from 0 mg to 20 mg per unit composition being administered,
esomeprazole in the composition is present in amount from 0 mg to 40 mg per unit composition being administered,
lansoprazole in the composition is present in amount from 0 mg to 30 mg per unit composition being administered,
pantoprazole in the composition is present in amount from 0 mg to 40 mg per unit composition being administered and
rabeprazole in the composition is present in amount from 0 mg to 20 mg per unit composition being administered.

The medicated chewing gum may include a gastroprotective agent such as an H2 antagonist present in doses of 0 mg to 150 mg per unit composition being administered.

The amounts of the active ingredient could be present as follows:
ranitidine in the composition is present in amount from 0 mg to 150 mg per unit composition being administered,
famotidine in the composition is present in amount from 0 mg to 40 mg per unit composition being administered and
cimetidine in the composition is present in amount from 0 mg to 150 mg per unit composition being administered.

The stick gum will be packaged for individual unit doses and allow for convenience to carry the product throughout the day. The packaging will be easy to open, a benefit over the blister packs commonly used for tablet gums today, given the arthritic state of the majority of target patients. The invention will be available in different flavors to help accommodate the fact that most patients who consume this product will need to do so chronically. The variety of flavoring will allow for the patients to have choice in terms of their tastes, without sacrificing efficacy. There will be different strengths of the components available in different packaging for different patient populations.

Referring to the figures, a stick of gum (10) is illustrated having two elements (11) and (12) separate by a calcium flavouring layer (13). Each of the parts 11 and 12 include a gum base and other typical ingredients found in a chewing gum stick and in addition an active GPA in 12 and an NSAID in 11. The GPA may be ranitidine and the NSAID ibuprofen. Typically the sticks of gum would be sold in a single box and each individual gum stick would be wrapped separately in paper wrappers thus making the product easy to use.

As many changes can be made to the various embodiments of the invention without departing from the scope thereof; it is intended that all matter contained herein be considered illustrative of the invention and not in a limiting sense.

While the foregoing provides a detailed description of a preferred embodiment of the invention, it is to be understood that this description is illustrative only of the principles of the invention and not limitative. Furthermore, as many changes can be made to the invention without departing from the scope of the invention, it is intended that all material contained herein be interpreted as illustrative of the invention and not in a limiting sense.

Embodiments of the invention in which an exclusive property or privilege is claimed are as follows:

1. A stick of medicated chewing gum comprising:
(a) a first part containing a gum base and a non-steroidal anti-inflammatory drug (NSAID);
(b) a second part containing a gum base and a protective amount of a proton pump inhibitor (PPI); and or
(c) a protective amount of H2 antagonists; and/or
(d) an acid pump antagonist.

2. The gum of claim 1 wherein the active ingredients are embedded within the first and or second parts separated by a calcium layer that contains a flavoring agent.

3. The gum of claim 2, further comprising an increased surface area to allow for easy absorption via the buccal mucosa, while minimizing contact between the medications.

4. The composition according to claim 3, wherein said stick of medicated chewing gum is formulated as a soft chewing stick to facilitate delivery and use by the affected population.

5. The composition of claim 2, wherein said flavoring agent is selected from the group consisting of menthol, organic acids, oil of cinnamon, oil of wintergreen, oil of peppermint, oil of spearmint, oil of sassafras, and oil of clove.

6. A composition according to claim 5 further comprising sweetening agents selected from the group consisting of sucrose, glucose, dextrose, levulose, saccharin salts, thaumatin, aspartame, D-tryptophan, dihydrochalcones, acesulfame salts cyclamate salts, sorbitol, xylitol, maltitol and mixtures thereof.

7. The composition of claim 1 wherein said NSAID is selected from the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts thereof, and mixtures thereof.

8. The composition according to claim 1 wherein said PPI is selected from a group consisting of lansoprazole, omeprazole, rabeprazole, esomeprazole, or pantoprazole and pharmaceutically acceptable salts thereof

9. The composition according to claim 1 wherein said H2 agonist is selected from the group consisting of ranitidine, cimetidine, or famotidine and pharmaceutically acceptable salts thereof.

10. The composition according to claim 1 wherein said acid pump agonist is selected from the group consisting of soraprazan, AZD0865, YH1885 and CS-526 and pharmaceutically acceptable salts thereof.

11. The composition according to claim 8 wherein the proton pump inhibitor is present in doses of 0 mg to 40 mg per unit composition being administered.

12. The composition according to claim 8 further comprising omeprazole in the composition present in amount from 0 mg to 20 mg per unit composition being administered.

13. The composition according to claim 8 further comprising esomeprazole in the composition present in amount from 0 mg to 40 mg per unit composition being administered.

14. The composition according to claim 8 further comprising lansoprazole in the composition is present in amount from 0 mg to 30 mg per unit composition being administered.

15. The composition according to claim 8 further comprising pantoprazole in the composition is present in amount from 0 mg to 40 mg per unit composition being administered.

16. The composition according to claim 8 further comprising rabeprazole in the composition is present in amount from 0 mg to 20 mg per unit composition being administered.

17. A composition according to claim 1 wherein the H2 antagonist is present in doses of 0 mg to 150 mg per unit composition being administered.

18. The composition according to claim 9 further comprising ranitidine in the composition is present in amount from 0 mg to 150 mg per unit composition being administered.

19. The composition according to claim 9 further comprising famotidine in the composition is present in amount from 0 mg to 40 mg per unit composition being administered.

20. The composition according to claim 9 further comprising cimetidine in the composition present in amount from 0 mg to 150 mg per unit composition being administered.
